(19) United States
(12) Patent Application Publication
(10) Pub. No.: US 2008/0182298 A1
(43) Pub. Date: Jul. 31, 2008

(54) Method And System For The Transformation Of Molecules,To Transform Waste Into Useful Substances And Energy

(76) Inventor: Andrew Eric Day, Longmeadow, MA (US)

Correspondence Address: Andrew Day;Eric Day, 325 Williams St., Longmeadow, MA, 01106, US

(21) Appl. No.: 11/627,403

(22) Filed: Jan. 26, 2007

(51) Int. Cl. A62D 3/02; C12P 19/00; C12P 3/00; G01N 37/00

(52) U.S. Cl. 435/72; 435/168; 435/262.5; 73/23.31

(57) ABSTRACT

The system, based on a recirculating Carbon Flow Loop, neutralizes toxins within municipal waste or other feedstock. A Plasma Syngas Gasifier is used to generate ultra high temperatures in an oxygen controlled atmosphere. This breaks down the feedstock into its basic elements, predominantly hydrogen and carbon monoxide, known as syngas. This can be used as a fuel, and/or be processed using water shift reaction, to yield additional hydrogen plus carbon dioxide. Following processing the carbon dioxide gas flow continues in the Carbon Flow Loop to an Algae Bioreactor. Here photosynthesis transforms it into oil rich algae. This can continue in the Carbon Flow Loop as feedstock for the Plasma Syngas Gasifier, and/or exit the loop, and be used to manufacture biofuels or other products. New feedstock is added to the Carbon Flow Loop to replace carbon lost or removed.

### FIELD OF INVENTION

The planet is being poisoned by toxic waste, while waste is not being put to useful work:

1. Carbon dioxide emissions from combustion engines, (used in power stations etc.) and rotting waste are creating global warming gases. This could contribute to destroying the planet, as we know it. The process may soon be irreversible.

2. Toxic waste from industrial factories and landfills is finding its way into our ground water supply.

3. Medical waste and dangerous bacteria need to be completely destroyed.

4. Landfills release methane into the atmosphere. Methane is 23 times more effective over a 100 year period at trapping heat as carbon dioxide.

5. Landfills and other waste streams are not being utilized as a resource.

The need to address these problems is urgent and compelling.

It is known that photosynthesis of algae creates carbohydrates by combining Carbon dioxide with water. Plasma Syngas Gasifiers break down substances to their basic elements by exposing them to the very high temperatures of an electric arc in ionized gas. Hydrogen engines release energy for useful work, and steam as an exhaust gas.

This invention is a system, which uses these processes and heat recovery techniques to form an efficient and practical way of cleaning up toxic waste and other refuse. By using landfills and other waste streams as a recoverable energy source we reduce our dependency on petroleum oil.

### BACKGROUND OF INVENTION

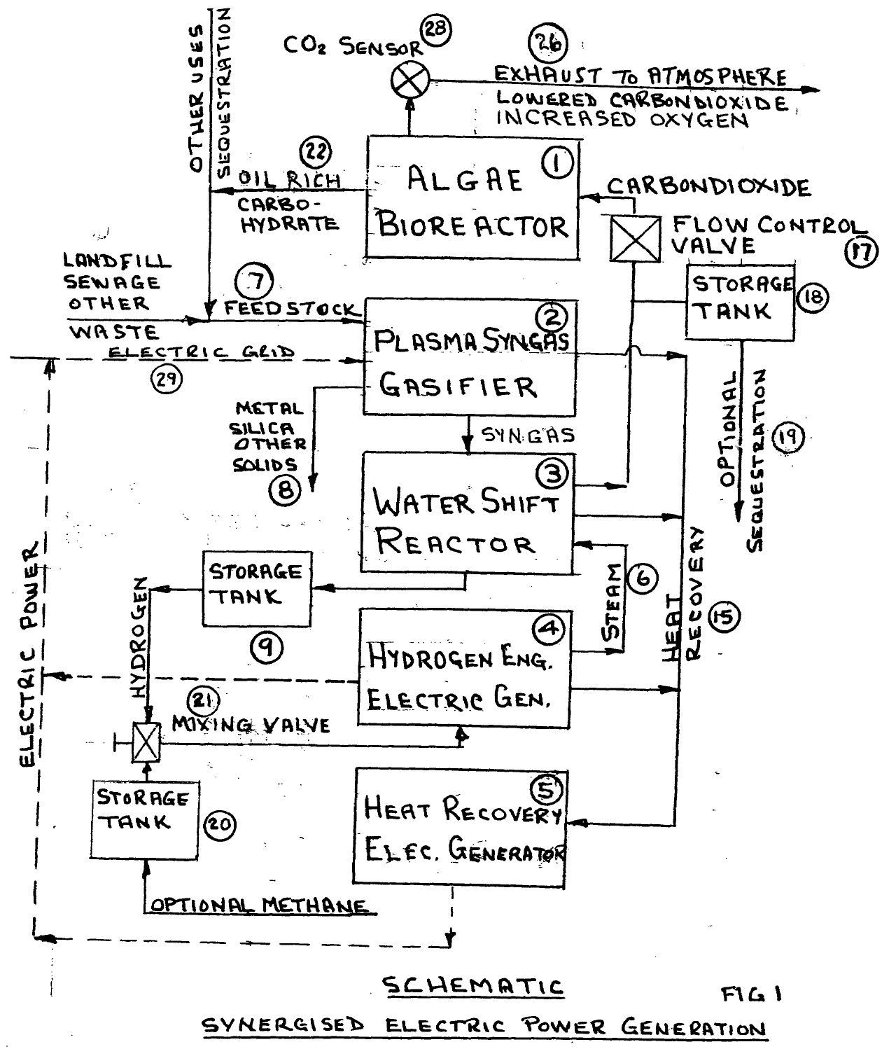

Building blocks for this system as shown in FIG. 1 are known:

1. Algae Bioreactors use fast growing algae, which in the presence of sunlight, feed on Carbon dioxide (CO₂), to become a valuable source of carbohydrate. Carbon dioxide is thus converted from a global warming pollutant into useful fuel feedstock rich in hydrogen, where 80% to 90% absorption is targeted

i.e.

Carbon Dioxide+Water+Plus sunlight=>Glucose+Water+Oxygen 6 CO₂+12 H₂O+Plus sunlight=>C₆ H₁₂ O₆+6 H₂O+6O₂

In general terms this resulting transformation is as follows:
Carbohydrate+Water+Oxygen
n CO+2nH₂+ATP+NADPH=>(C H₂O)n+n H₂O+nO₂
Where n is defined according to the structure of the resulting carbohydrate,
ATP is adenosine triphosphate,
NADPH is nicotinamide adenosine dinucleotide phosphate.

Whereas hydrocarbons are typically defined as: CnH₂n+₂. They lack oxygen.

2. Plasma Syngas Gasifiers can achieve temperatures hotter than the sun's surface, by striking an electric arc through ionized gas, in much the same way as a lightning bolt. At these elevated temperatures, molecules within compounds are transformed into their basic elements. Hydrocarbons and carbohydrates are split into carbon monoxide and hydrogen. Base metals and silica etc. form part of a molten discharge. These can be drained off to solidify on cooling. The non- precious slag can be used as a building material for industrial products.

i.e. Hydrocarbon and Carbohydrate Feedstock+Heat Absorption→Syngas Syngas, is comprised of mainly carbon monoxide CO and hydrogen H

3. Water Shift Reactors are used to combine high temperature steam with the syngas. This combines oxygen from the steam with carbon monoxide from the syngas to become carbon dioxide. The remaining hydrogen is bled off.

i.e.: Syngas+Steam=>Carbon dioxide+Hydrogen CO+H₂+H₂O=>CO₂+2H₂

4. Hydrogen engines ignite the hydrogen gas in the engine combustion chamber and can be used to drive an electric generator or other devices. The exhaust from this process is steam, which can be fed directly to the Water Shift Reactor, or after recovering heat energy, used as clean hot water.

i.e. Hydrogen+Oxygen+Heat Release=>Steam 2H₂+O+Heat Release=>2H₂O

5. Heat Recovery from the Plasma Syngas Gasifier (Item 2) the Plasma Syngas Gasifier molten discharge (Item 8), the Water Shift Reactor (Item 3), and the Hydrogen Engine Electric Generator(Item 4) can be used for many industrial processes, including powering a refrigerant turbine to power an electric generator. These units use waste heat to evaporate refrigerant gas. This is used to power a low temperature gas turbine engine (part of Item 5 FIG. 1), which drives a generator, and is used to supplement the electric power provided by the Hydrogen Engine Electric Generator.

### OBJECT OF INVENTION

Is to provide a means of controlling the greenhouse gas emissions to atmosphere, while generating electricity and/or producing oil rich carbohydrates (algae) and hydrogen gas. The feedstock used being hydrocarbons, carbohydrates, sewage or other feedstock.

### SUMMARY OF INVENTION

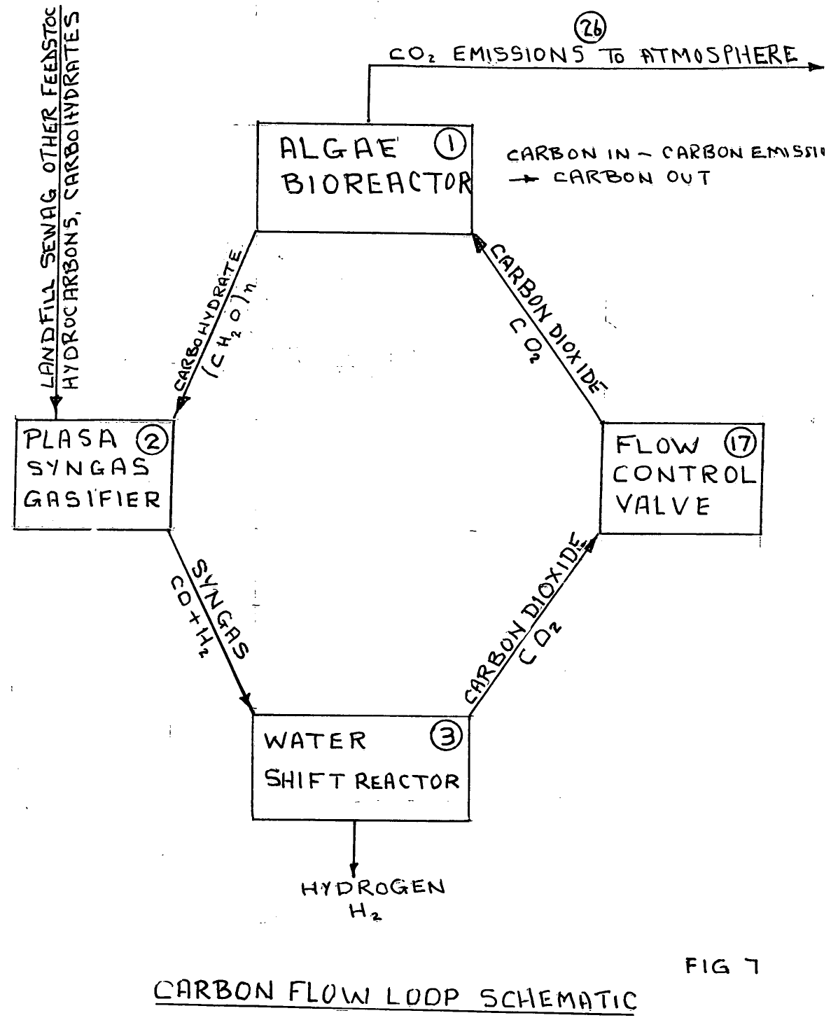

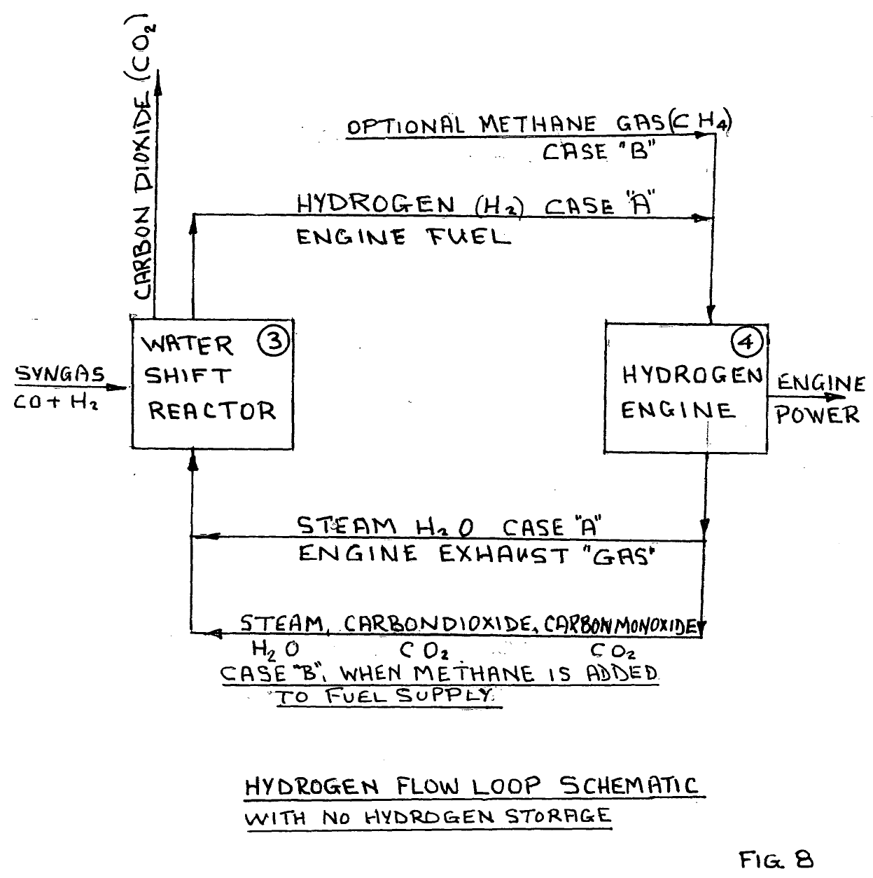

The system shown in FIGS. 7, and 8, contains two flow loops, one carbon and the other hydrogen:

Carbon Loop

In the Carbon Flow Loop shown in FIG. 7, the Algae Bioreactors (Item 1) gathers and supplies carbohydrates. This may be fed either to the feedstock input of the Plasma Syngas Gasifier (Item 2), or put to other uses, or sequestration. Other hydrocarbon/carbohydrate feedstocks can also be fed to the Plasma Syngas Gasifier. From this input it supplies syngas (CO+H) to the Water Shift Reactor (Item 3), which supplies Carbon dioxide back to the Algae Bioreactor (Item 1) via Flow Control Valve (Item 17).

Hydrogen Loops

In “Case A” Hydrogen Flow Loop, shown in FIG. 8, the Water Shift Reactor (Item 3), supplies hydrogen gas to the Hydrogen Engine Electric Generator (Item 4). Combustion within the Engine combustion chamber creates steam, which is fed back to the Water Shift Reactor to close the loop. Water gas shift reaction within Water Shift Reactor strips the oxygen atom from the steam (H₂O) and adds them to the carbon monoxide (CO) to become carbon dioxide (CO₂), the released hydrogen (H₂) is then fed back to the Hydrogen Engine. In “Case B”, methane (CH₄), is mixed with the hydrogen from the Water Shift Reactor (ref. Fig. 1 and 8, Item 3) and fed to the Hydrogen Engine (ref Fig. 1 and 8 Item 4). Combustion within the Engine creates steam, carbon dioxide and possibly carbon monoxide. The engine exhaust is fed back to the Water Shift Reactor, where the carbon dioxide will pass through it and become part of the Carbon Flow Loop. In the case of carbon monoxide, this will become carbon dioxide and then also become part of the Carbon Flow Loop. The sources of the carbon gases are the optional use of methane to supplement the hydrogen fuel supply and any carbon gases present in the oxygen supply to the Hydrogen Engine. This carbon plus the carbon added in the Feedstock (Item 7) are both addition to the carbon flowing in the carbon flow loop.

### BRIEF DESCRIPTION OF DRAWINGS

Item 1. Algae Bioreactors, (ref. FIG. 1 through 6). Photosynthesis of the algae in the presence of sunlight creates an oil rich carbohydrate, by combining carbon dioxide with water. Carbon dioxide is thus converted from a global warming pollutant into a useful energy source. Surplus oxygen and any undigested carbon dioxide is vented to atmosphere.

Item 2. Plasma Syngas Gasifier, (ref FIG. 1 through 6). Ionized gas known as plasma is a good conductor of electricity. A continuous electric arc struck within the plasma can produce temperatures greater than 30,000 degrees Fahrenheit (F). Within an oxygen depleted atmosphere at these temperatures both hazarded and non-hazardous materials in the feedstock are broken down into their basic elements. This is known as sygas. Municipal solid waste feedstock comprising typically of carbohydrates CH₂O and hydrocarbons CH₂, breaks down into carbon dioxide CO₂ and hydrogen H₂, with typically up to 10% other gases.

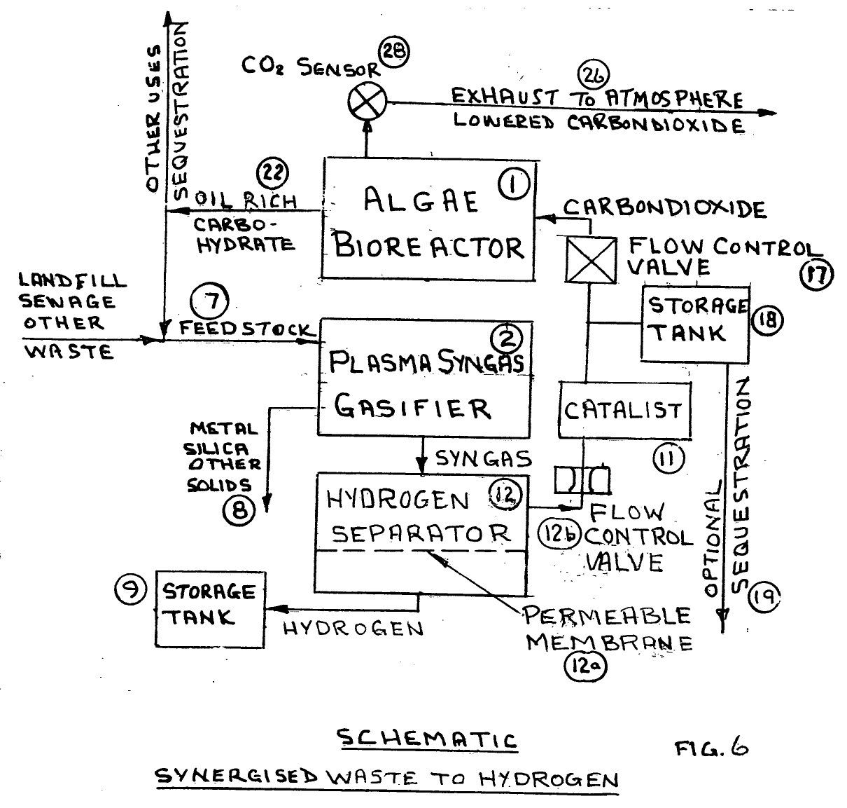

Item 3. Water Shift Reactors, (ref. FIGS. 1 through 4), are used to combine the oxygen atoms in hot steam (H₂O) with carbon monoxide (CO) to become carbon dioxide (CO₂) and release the remaining (H₂) atoms as hydrogen gases. To separate the lighter hydrogen gas (atomic weight 1) from the carbon dioxide (atomic weight 44), the lighter hydrogen is drawn from the top of a temporary storage tank and the Carbon dioxide from the bottom. If purer hydrogen is required it can be passed through item 12, the Hydrogen Separator (ref. FIG. 6).

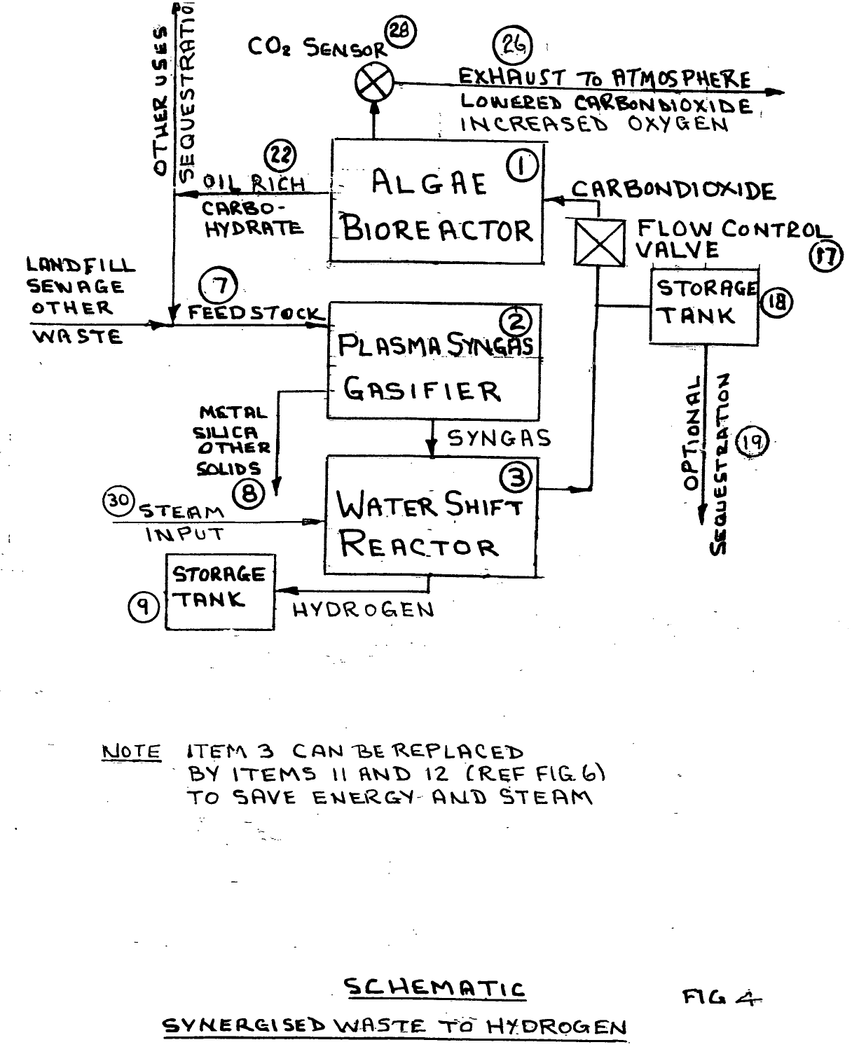

Item 4. Hydrogen Engines Electric Generators, (ref. FIG. 4), is an internal combustion engine which ignites hydrogen or a mixture of hydrogen and methane (natural gas with oxygen to drive an electric generator.

Item 5, Heat Recovery Electric Generator, (ref. FIG. 1, 2, and 3). Recovered waste heat, item 15, is used to boil refrigerant gas, which provides power to a low temperature gas turbine engine. This is used to drive an electric generator.

Item 6, Steam (ref FIG. 1 through 3). Hot steam is fed to the Water Shift Reactor.

Item 7, Landfill Sewage Other Waste, (ref. FIG. 1 through 6), is the primary feedstock used by these systems. Other hydrocarbon or carbohydrate based waste could be tires, used engine oil or high energy industrial waste.

Item 8. Metals Silica and Other Solids, (ref. FIG. 1 through 6), which do not gasify, drain off as molten discharge.

Item 9, Hydrogen Storage, (ref. FIG. 1, through 4, and FIG. 6), provides a means of storing hydrogen for later use.

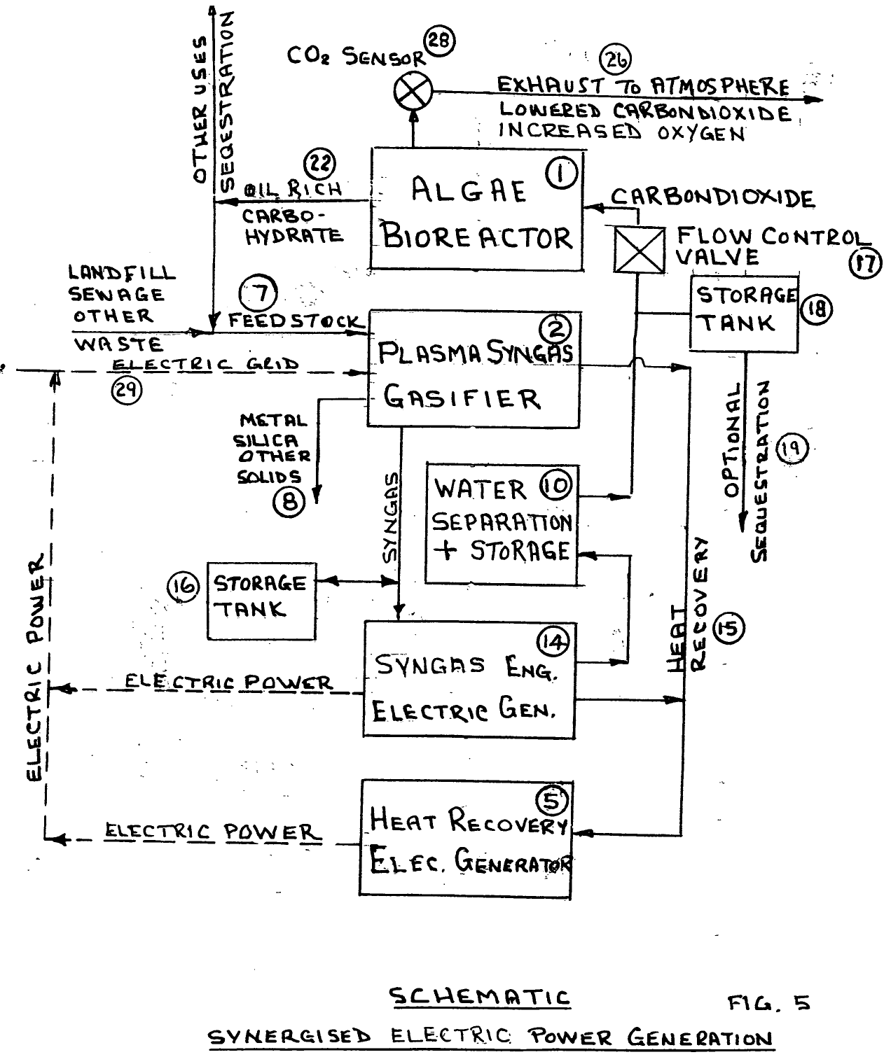

Item 10, Water Separation and Storage Unit, (ref. FIG. 5). During combustion of the syngas, Carbon dioxide and steam are formed. Heat transfer from the (Syngas Engine) exhaust gas, to the Heat Recovery Circuit (Item 15) will lower the steam temperature to below boiling point. The storage tank will now contain water at the bottom and Carbon dioxide above it.

Item 11, Catalytic Converter. (ref FIG. 6), converts any carbon monoxide present in the Hydrogen Separator exhaust (Item 12) into Carbon dioxide for digestion by the Algae Bioreactor. Heat generated by this process can be used to dry feedstock when needed or put to other Heat Recovery (Item 15) uses,

Item 12, Hydrogen Separator. (Ref. FIG. 6)

Item “12a” is a fine porous membrane that allows hydrogen to pass through it, but not larger molecules such as carbon dioxide.

Item “12b” Flow Control Valve maintains a constant pressure drop across the membrane to control the proportion of hydrogen separated.

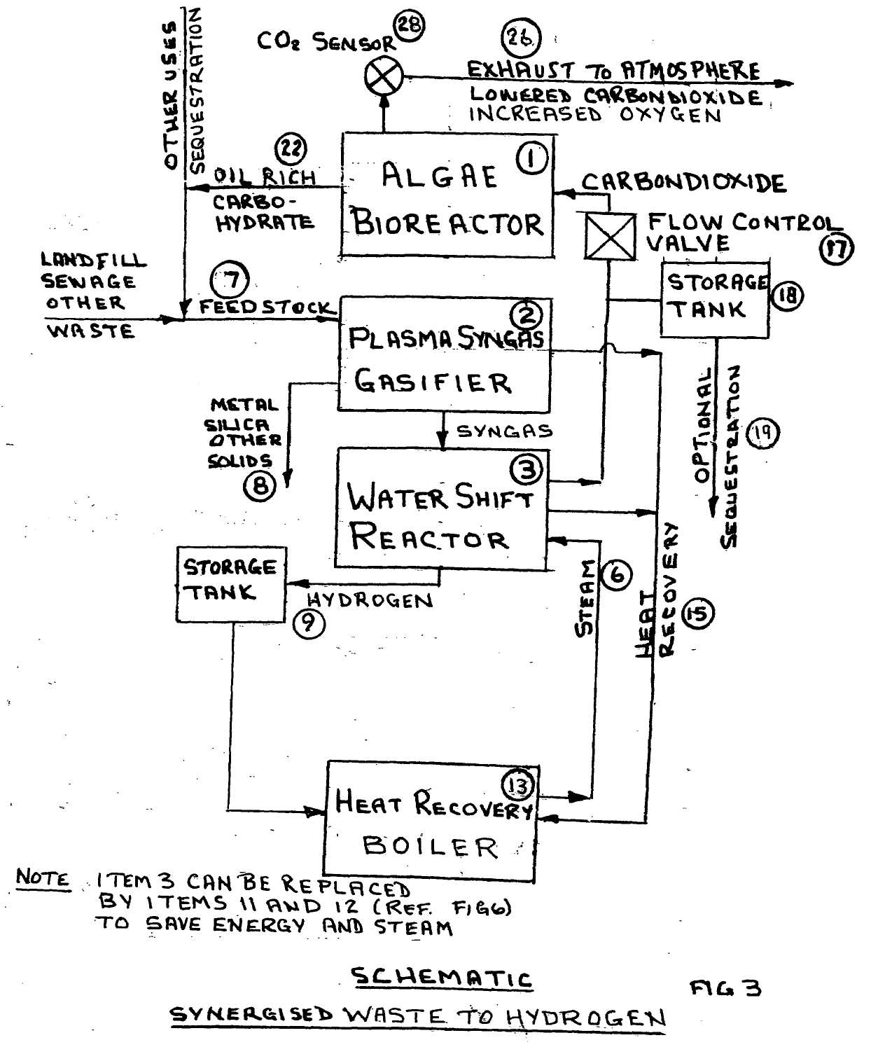

Item 13, Heat Recovery Boiler, (ref FIG. 3), uses the Heat Recovery fluid, item 15, to preheat the water input to the boiler. Following this the water is boiled into hot steam by the combustion hydrogen fed from the Water Shift Reactor

Item 14, Syngas Engine, (ref. FIG. 5), is an internal combustion engine, which ignites syngas (carbon monoxide and hydrogen) with oxygen in the engine combustion chamber. It is used to drive an electric generator. The exhaust “gases” from this process are carbon dioxide, steam and possibly some carbon monoxide.

Item 15, Heat Recovery, (ref. FIG. 1, 2, 3, and FIG. 5). Heated fluid (Item 15), is supplied by the Plasma Syngas Gasifier (Item 2), the Water Shift Reactor (Item 3), and either Hydrogen Engine (Item 4), or Syngas Engine (Item 14).

Item 17, Flow Control Valve, (ref. FIGS. 1 through 7), uses the input from the CO₂ Sensor (Item 28), to control the flow of carbon dioxide to the Algae Bioreactor (Item 1). By avoiding over supply, greenhouse gas emissions from the Algae Bioreactor to atmosphere (Item 26) are limited to a preset value

Item 19, Sequestration, (ref FIG. 1 through 6), is the optional possibility to store the carbon dioxide elsewhere.

Item 20, Methane Storage Tank, (ref FIG. 1), is a holding tank to enable flow restriction of gas flow by Mixing Valve (Item 28). Methane is the main constituent of Natural Gas. This can also be used.

Item 21, Hydrogen/Methane Mixing Valve (ref FIG. 1), is the valve controlling the percentages of Hydrogen and Methane being fed to the “Hydrogen Engine”. Methane is the main constituent of Natural Gas (ref. previous continuation patent application Ser. No. 11/624,240).

Item 22, Oil Rich Carbohydrate, (ref FIG. 1 through 6), is the product harvested by the Algae Bioreactor.

Item 26, Bioreactor Exhaust Gas, (ref. FIGS. 1 through 6), is vented to atmosphere. The initial targeted digestion rate of carbon dioxide by the algae is 80% to 90%. The 10% to 20% of carbon dioxide being released will also contain additional oxygen This is released during photosynthesis of the carbon dioxide input and water.

Item 28, Carbon Dioxide Sensor, (ref FIG. 1 through 6), is used to measure the quantity of carbon dioxide gas being emitted to atmosphere by the Algae Bioreactor.

Item 29, Electric Grid, (ref FIG. 1 through 6), can receive power from the facility, or supply power to the facility.

Item 30, Clean Steam Supply, (ref. FIG. 4) is used when clean steam is available. Capital costs can be reduced by omitting the Hydrogen Engine Electric Generator item 4, the Heat recovery Electric Generator item 5, and the Heat Recovery System item 15.

### DESCRIPTION OF PREFERRED EMBODIMENT

The greenhouse gas emission flowing to atmosphere (Item 26) can be controlled by a closed loop feedback control system, where measurement of variances by the CO₂ Sensor (Item 28) from the targeted CO₂ emissions can be fed back to the Flow Control Valve (FIGS. 1 thru 6, Item 17) and the supply of CO₂ fed to the Algae Bioreactor (Item 1) continuously adjusted. To limit the build up of Carbon dioxide in Storage Tank (Item 18), the energy input to the Plasma Syngas Gasifier electric arc also needs to be adjusted. If necessary, increased feedstock flow rates could be achieved by sequestration of carbon dioxide via the Storage (Item 19).

The amount of carbon flowing in the Carbon Flow Loop is controlled the syngas output of the Plasma Syngas Gasifier, since after adding oxygen this determine the amount of carbon dioxide fed to the Algae Bioreactor via Flow Control Valve (Item 17). For the Plasma Syngas Gasifier to supply carbon monoxide and hydrogen (syngas) the supply of oxygen needs to be carefully controlled. Additional oxygen in the form of air, steam or water finding its way into the Plasma Syngas Gasifier increases the formation carbon monoxide or produces carbon dioxide when free carbon is not available. With this sensitivity, the dryness of the feedstock can be seen to be critical, and need good process control. Cyclone dryers and other ways to evaporate moisture may need to be employed for this. Carbohydrate feedstocks are more sensitive to this problem since their makeup includes oxygen atoms, whereas hydrocarbons do not

As can be seen from FIG. 7.

The Algae Bioreactor carbon balance is as follows:

Algae Bioreactor input carbon−carbon to atmosphere=Algae Bioreactor output carbon. in carbon dioxide in carbon dioxide in carbohydrate (algae) For steady system flow, the carbon in the carbon dioxide emissions to atmosphere (Item 26), and any other carbon particles removed from the system, would need to be replaced by adding feedstock (Item 7) to the Plasma Syngas Gasifier. For example, if all the carbohydrate from the Algae Bioreactor (Item 22) were fed to the Plasma Syngas Gasifier (Item 2), and no carbon was removed from the system, the only added feedstock would be that with the same carbon content as the carbon dioxide emissions (Item 26). If the added feedstock were only carbohydrate, more oxygen may not need to be fed to the Plasma Syngas Gasifier. if the carbohydrate contains matching carbon and oxygen atoms, however, if hydrocarbon feedstock (with no oxygen content) were added, more oxygen would be required. On the other hand if the oxygen supply to the Plasma Syngas Gasifier is insufficient to transform all the carbon atoms into carbon monoxide. Unbonded carbon would remain as carbon black. This would either drain from the Plasma Syngas Gasifier with other solids or could be filtered out from cooled syngas. In the case where excess moisture in the feedstock (Item 7), creates the need to reduce the oxygen level in the Plasma Syngas Gasifier, this could possibly be done by using a dry source of hydrocarbon feedstock (Item 7) such as dry used tires.

Variations on this proposal can be made to suit specific application.

These are shown on FIGS. 1 through 6.

FIG. 1. This is the base design. Optional configurations are listed below:

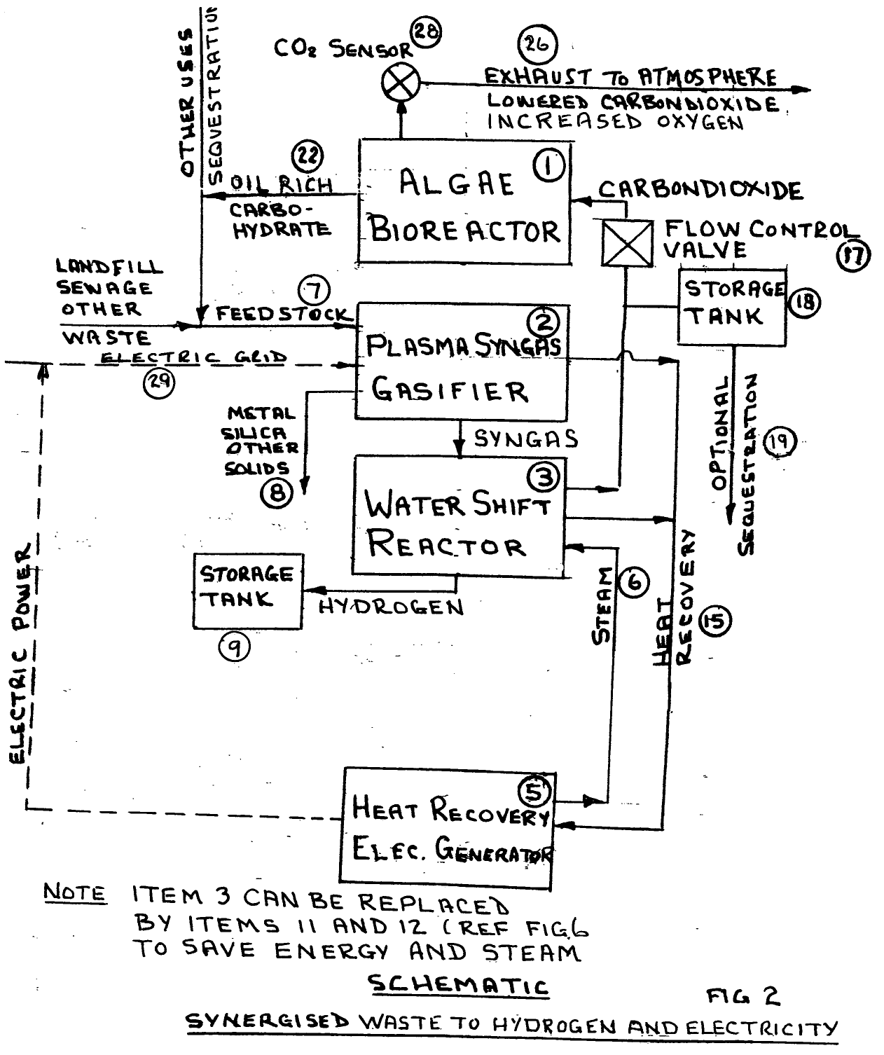

FIG. 2. Less electricity, more hydrogen, lower cost

FIG. 3. No electricity, even more hydrogen, even lower cost

FIG. 4. No electricity, similar hydrogen, no heat recovery, no steam supply from Hydrogen Engine Electric Generator (Item 4) to Water Shift Reactor (Item 3).

FIG. 5. No hydrogen production, more electricity

FIG. 6. No electricity, no heat recovery, even lower cost

As shown on FIG. 1, carbohydrate from the Algae Bioreactor (Item 1), and carbohydratelhydrocarbon from landfills, sewage or other feedstock (Item 7) can be fed to the Plasma Syngas Gasifier (Item 2) to produce syngas. This is then fed to the Water Shift Reactor (Item 3), where with steam input (Item 6), the carbon monoxide is converted into carbon dioxide and fed back to the Algae Bioreactor (Item 1). Hydrogen is also fed to the Hydrogen Engine Electric Generator (Item 4) and Hydrogen Storage Tank (Item 9). With adequate hydrogen storage the Hydrogen Engine Electric Generator (Item 4) becomes an uninterrupted source of electricity. It is also used to provide hot engine water to the Energy Recovery System (Item 15). The engine exhaust is steam, which is fed directly to the Water Shift Reactor, where its oxygen component combines with carbon monoxide (in the syngas) to become carbon dioxide and the hydrogen gas is released

Heat can also be recovered from the Plasma Syngas Gasifier Molten Discharge (Item 8), and the Plasma Syngas Gasifier and Water Shift Reactor cooling jackets. To improve overall operating efficiency, the recovered heat can be used to evaporate refrigerant gas, to power a low temperature gas turbine engine (ref. Item 5). This drives a generator, which supplements the electric power provided by the Hydrogen Engine Electric Generator (Item 4). Byproducts of the Plasma Syngas Gasifier (Item 2) operation are the recycled base metals, silica, and other solids, which melt and form part of a molten discharge (Item 8). In cases where methane gas is being emitted from landfills or other feedstock sources, it can be used as a fuel for the Hydrogen Engine. As shown in (FIG. 1) the methane is fed to Storage Tank (Item 20), then to Mixing Valve (Item 21) where hydrogen gas and/or methane gas can be fed to the Hydrogen Engine (Item 4).

As shown on the embodiment in FIG. 2, the FIG. 1 system is modified to omit item 4, the Hydrogen Engine Electric Generator. This embodiment is better suited for applications where more hydrogen is required (to be stored in item 9) as the final product. Supplemental heat may be required to boil the heat recovery water into steam (Item 6). This embodiment reduces the electric power, which can be supplied to the electric grid, but also reduces the initial capital cost of the system.

As shown on the embodiment in FIG. 3, the FIG. 1 system is modified to omit item 4, the Hydrogen Engine Electric Generator, and item 5, the Heat Recovery Electric Generator. This is replaced by item 13, a Heat Recovery Boiler. This embodiment is suited for applications where only hydrogen is required (to be stored in item 9) as the final product. This embodiment does not provide any electric power to the electric grid, but reduces the initial capital cost of the system.

As shown on the embodiment in FIG. 4, the FIG. 1 system is modified to omit item 4, the Hydrogen Engine Electric Generator, item 5, the Heat recovery Electric Generator, and the Heat Recovery System, item 15. It omits steam injection from the Hydrogen Engine Electric Generator (Item 4) into the Water Shift Reactor. This needs to be replaced by another clean steam source. This further reduces the initial capital cost of the system. This embodiment is suited for applications where only hydrogen is required (to be stored in item 9) as the final product. This embodiment does not provide any electric power to the electric grid, but reduces the initial capital cost of the system.

As shown on the embodiment in FIG. 5, the FIG. 1 system is modified to omit item 3, the Water Shift Reactor, and item 4, the Hydrogen Engine Electric Generator. These are replaced by item 14 the Syngas Engine Electric Generator, and item lo the engine exhaust gas Water Separator And Storage unit. This embodiment generates electricity but does not provide any hydrogen gas. It reduces the initial capital cost of the system.

As shown on the embodiment in FIG. 6, the FIG. 1 system is modified to omit item 3 the Water Shift Reactor, item 4 the Hydrogen Engine Electric Generator, item 5 the Heat Recovery Electric Generator, and item 15 the Heat Recovery System. These are replaced by item 12 the Hydrogen Separator, and item 11 the Catalyst. The Hydrogen Separator, item 12, incorporates a Hydrogen Permeable Membrane (Item 12a), which allows the small Hydrogen molecules to pass through it, and Flow Control Valve (Item 12b). The rest of the Syngas flows to the Catalyst where carbon monoxide is converted into Carbon dioxide.

This is then fed back to the Algae Bioreactor to continue the cycle. This embodiment provides hydrogen but not electric power and further reduces the initial capital cost of the system.

It will be apparent to a person of ordinary skill in the art, that various modifications and variations can be made to the system for operating the generating system without departing from the scope and spirit of the invention. It will also be apparent to a person of ordinary skill in the art that various modifications and variations can be made to the size and capacity of the items listed from 1 to 30 shown on FIGS. 1 through 6 without departing from the scope and spirit of this invention. Thus it is intended that the present invention cover the variations and modifications of the invention, providing they come within the scope of the appended claims and their equivalents.

1. It is the object of this invention to provide a method and system to remove carbon black from hydrocarbon fuel and harvest the remaining hydrogen.

2. It is the object of this invention is to provide a method and system, to modulate hydrocarbon and/or carbohydrate feedstock inputs to the Plasma Syngas Gasifier, in order to control the amount of carbon dioxide in the Carbon Flow Loop.

3. It is the object of this invention is to provide a method and system, to remove carbon black from hydrocarbon feedstock to increase the removal of landfill sewage or other waste.

4. It is the object of this invention is to provide a method and system, to continuously monitor and regulate Carbon dioxide emissions to atmosphere while generating electrical power and/or harvesting hydrogen gas.
